(19) United States
(12) Patent Application Publication
(10) Pub. No.: US 2005/0251103 A1
(43) Pub. Date: Nov. 10, 2005

(54) Method and device for displaying and metering doses as a product is administered

(76) Inventors: Beat Steffen, Saanen (CH); Julian Yeandel, Grosshoechstetten (CH); Alessandro Sebaste, Basel (CH); M'hamed Moulay Heniche, Burgdorf (CH); Alain Charles Boss, Bellach (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/119,107

(22) Filed: Apr. 29, 2005

Related U.S. Application Data
(63) Continuation of application No. PCT/CH03/00693, filed on Oct. 24, 2003.

(30) Foreign Application Priority Data
Oct. 30, 2002 (DE) 102 50 629.9

(51) Int. Cl. A61M 31/00

(52) U.S. Cl. 604/500; 604/207

(57) ABSTRACT

A device and method for displaying and metering a product to be administered from a reservoir by means of a dose metering device and a display unit, wherein a specific dose is set by moving the dose metering unit from an initial position in a metering direction across a dose metering distance. A reference point is defined by an initial position of the dose metering device in readiness for administering a first dose or a position assumed by the dose metering device after administering a dose. The dosage setting displayed by the display unit is a dosage value based on the distance travelled by the dose metering device from the reference point in the metering direction.

### CROSS-REFERENCE TO RELATED APPLICATIONS

This application is a continuation of International Application No. PCT/CH2003/000693, filed on Oct. 24, 2003, which claims priority to German Application No. 102 50 629.9, filed on Oct. 30, 2002, the contents of which is incorporated in their entirety by reference.

### BACKGROUND

The present invention relates to devices and methods for administering, dispensing, delivering or injecting substances and, more particularly, to a method and a device for displaying and metering doses of a product or substance administered from a reservoir, whereby a desired dose to be administered may be set or selected by using a dose metering device.

Administering devices such as injection pens, for example, are used as a means or tool for administering a metered quantity of a product, such as a fluid product for medicinal, pharmaceutical or cosmetic purposes. Typically, such administering devices have a reservoir, for example an ampoule, in which the product is contained. The product is administered from the reservoir through an outlet, by means of a plunger disposed in the reservoir, which is displaced in the direction towards the outlet by a drive mechanism by operating a drive button. A dose metering device is usually provided as a means of metering doses of the product to be administered, which controls the displacement of the drive mechanism when the drive button is operated, depending on a desired dose. Accordingly, the dose metering device restricts the distance which a drive mechanism is able to travel when the drive button is operated and hence also the distance by which the plunger is displaced inside the reservoir in the direction towards the outlet. To this end, the dose metering device can be displaced, e.g., is rotatable or slidable, in a metering direction relative to a housing of the injection pen, for example. In order to set a specific dose, the dose metering device is moved from an initial position in the metering direction by a dose metering distance which corresponds to the desired dose and which determines a distance by which the plunger has to be moved forward in order to administer the desired volume.

In some known devices, an electric display on the administering device records the displacement of the dose metering device and displays a selected dose. This being the case, the dosage value is displayed on the basis of the dose metering distance travelled by the dose metering device in the metering direction from the initial position. To this end, the displacement of the dose metering device may be operated on the basis of a catch mechanism, for example, whereby each catch position corresponds to a dosage unit. When the dose metering device is moved from the initial position in the metering direction, the display is incremented by one dosage unit each time a catch position is overcome. Once the selected dose has been administered, the display can be re-set to nil or its initial setting with the aid of a re-set button.

If, however, the dose metering device is moved across a distance in the direction opposite the metering direction, i.e., in the metering counter-direction, the result of this distance as far as the drive mechanism is concerned is a zero-dosage distance. When the drive mechanism and the plunger are then moved in the forward direction towards the outlet, no product is dispensed across this zero-dosage distance. This is generally referred to as a “dry shot”.

When the dose metering device is moved in the metering counter-direction after administering a dose, i.e., across a zero-dosage distance, a warning appears on the display. The display can be turned back to the nil value by moving the dose metering device in the metering direction, so that the dose metering device is returned to the original position after administering a dose. However, there is a possibility that a user might ignore this warning and operate the re-set button in spite of this warning, in which case the nil value is displayed on the display even though the dose metering device is in a position it assumed after effecting a zero-dosage distance. As soon as a first movement is effected in the metering direction and a first catch position is overcome, the dose display is incremented. However, the zero-dosage distance is also included in the count for a desired dose as a dose metering distance. With the next administering process is initiated with the aid of the drive mechanism, no product is dispensed on this zero-dosage distance when the plunger is moved forward, which means that the product quantity is short by a quantity of product corresponding to the zero-dosage distance, even though the display indicates the correct dosage. When the product is administered, the dose is therefore short, which can impair the effect which the product is intended to produce.

In the prior art, this problem has been overcome by alerting the user to this problem by providing a warning in an instruction manual accompanying the administering device. The user is told that if a corresponding warning or prompt is shown on the display after a dry shot, he must prime the administering device manually. Priming involves setting a dose of one unit, for example, and depressing the drive button and then the re-set button in order to set the display to nil. As the drive button is depressed, a drop of product is forced out of the outlet of the reservoir and the device is ready to perform the administering process. If no drop appears, the process must be repeated. During this procedure, the drive mechanism travels the zero-dosage distance. When the dose metering device is moved in the metering direction after priming and covers a dose metering distance, the full desired dose is forced out during the next administering process and the dose is not short. However, this priming procedure is inconvenient for the user as well as time-consuming and can easily be forgotten if the warning is not seen or is ignored, for example.

### SUMMARY

Accordingly, one objective of the present invention is to provide a method of displaying and metering a product as it is administered, which substantially ensures that a correct dose can be set and administered, minimizes the effort required on the part of the user when metering and administering a dose, and automatically corrects the display in the event of incorrect operation.

Thus, in one embodiment, the present invention comprises a method of administering a substance from an administering device comprising a reservoir containing the substance, a dose metering unit and a display unit, the method comprising the steps of selecting a dose of the substance to be administered by moving the dose metering unit from an initial position in a metering direction across a dose metering distance, defining a reference point by an initial position of the dose metering unit in readiness for administering a first dose or a position assumed by the dose metering unit after administering a selected dose, and displaying the selected dose on a display unit associated with the administering device wherein the displayed dose value is based on the distance travelled by the dose metering unit from the reference point in the metering direction.

In one embodiment, the present invention comprises a method of displaying and metering doses of a product to be administered from a reservoir by means of a dose metering device and a display unit, whereby, in order to set a specific dose of the product, the dose metering device is moved starting from an initial position (NP_{abs}, A) in a metering direction across a dose metering distance (1a; 1b; 3a, 3a′; 3b, 3b′), and wherein a reference point (NPᵣₑₗ) is defined by a position of the dose metering device assumed after administering a dose or by the initial position (NP_{abs}) of the dose metering device in readiness for a first dose to be administered and a dosage setting is displayed as a dosage value on the basis of the distance (3b′) travelled by the dose metering device from the reference point (NPᵣₑₗ) in the metering direction.

Another objective of the invention is to provide a device for administering metered doses of a product, which is simple to use, substantially ensures that a desired dose is correctly set and administered, and automatically displays a correct dose to reflect the actual dosage value that has been set in the event of incorrect operation.

Thus, in one embodiments, the present invention comprises a device for administering metered doses of a product, comprising a reservoir for the product, a dose metering device, which dose metering device is displaceable starting from an initial position (NP_{abs}, A) in a metering direction along a dose metering distance (1a; 1b; 3a, 3a′; 3b, 3b′) in order to set a specific dose of the product and a display unit for displaying a set dosage value, wherein the display unit displays a dosage value on the basis of a distance (3b′) travelled by the dose metering device in the metering direction, which distance (3b′) is determined from a reference point (NPᵣₑₗ) constituting a position assumed by the dose metering device after administering a dose (NPᵣₑₗ) or an initial position (NP_{abs}) of the dose metering device in readiness for a first dose to be administered.

In one embodiment, the present invention comprises a device and method for displaying and metering a product to be administered from a reservoir by means of a dose metering device and a display unit, wherein a specific dose is set by moving the dose metering unit from an initial position (NP_{abs}, A) in a metering direction across a dose metering distance (1a; 1b; 3a, 3a′; 3b, 3b′). A reference point (NPᵣₑₗ) is defined by an initial position of the dose metering device in readiness for administering a first dose or a position assumed by the dose metering device after administering a dose. The dosage setting displayed by the display unit is a dosage value based on the distance (3b′) travelled by the dose metering device from the reference point (NPᵣₑₗ) in the metering direction.

For the purposes of the invention, a dose metering device and a display unit are used to operate a method of displaying and metering a product or substance to be administered from a reservoir, whereby the dose metering device is moved from an initial position in a metering direction across a dose metering distance in order to set a specific dose. A reference point is defined on the basis of an initial position assumed by the dose metering device in readiness for administering a first dose or a position assumed by the dose metering device after administering a dose. When a dose is set, the dosage value of this dose is indicated by the display unit on the basis of the distance which the dose metering device has travelled in the metering direction from the reference point. Accordingly, the reference point constitutes a nil point, from which the distance covered by the dose metering device in the metering direction is measured when setting the dose. This is effectively a relative nil point because this nil point is newly defined whenever a dose is administered. The reference point for a subsequent administering process is therefore shifted by a distance which the dose metering device has travelled in the metering direction since the reference point of the administered dose setting.

For example, in accordance with the present invention, if the metering device was moved in the dose counter-direction after dispensing a dose, i.e., was moved across a zero-dosage distance, this zero-dosage distance is taken into account when the dosage value is displayed and the correct value of the set dose is displayed. This being the case, the value displayed for the dose setting is effectively a dose metering distance travelled by the dose metering device in the metering direction less the zero-dosage distance. n other words, if a user moves the dose metering device from an initial position across a dose metering distance in the metering direction, the distance which the dose metering device travelled in the direction opposite this direction before being moved in the metering direction is subtracted. Allowance is therefore automatically made for incorrect operation in the metering counter-direction when a set dosage value is displayed.

In some embodiments, the reference point and relative nil point are preferably indicated by the display unit in the form of a code. The display “00” is selected for this purpose, for example. If a user moves the dose metering device across a distance in the metering counter-direction, i.e., a zero-dosage distance is set, a code is preferably used by the display unit to represent this procedure, for example in the form of two minus signs, as a warning to the user. If the dose metering device is then moved across a distance in the metering direction, the code representing the reference point will not appear on the display until the dose metering device has moved past the zero-dosage distance in the metering direction again and the reference point or relative nil point is reached. From this point onwards, the display starts to indicate the dosage value as the dose is being set.

In accordance with one embodiment of the method proposed by the invention, an absolute nil point is preferably defined on the basis of specific positions of the dose metering device. The absolute nil point may be defined, for example, by the position which the dose metering device assumes when a reservoir is fitted on the metering device, i.e., when an ampoule is replaced or newly inserted in an administering device, and if necessary re-set to nil. Furthermore, the absolute nil point can be manually defined by the user, irrespective of the position of the dose metering device, by re-setting the display unit to nil, i.e., by a priming routine. As a dose metering device is moved back from a forward position with the plunger pushed towards the outlet into a rear position in the administering device, i.e., into a start position in readiness for setting subsequent doses, a larger distance has to be covered by the dose metering device in the metering counter-direction. This being the case, an absolute nil point can be defined by the position assumed by the dose metering device after it has travelled a pre-defined minimum distance in the metering counter-direction, in which case the display unit registers a reverse distance travelled and the distance is preferably significantly greater than a dosage distance. For example, the position which the dose metering device assumes after moving back by more than 15 dosage units can be defined as an absolute nil point, after which a priming routine is performed. Finally, a position assumed by the dose metering device when it is pulled back as far as possible in the administering device, i.e., when the dose metering device is disposed on a rear stop in the administering device, can be defined as an absolute nil point. To this end, a suitable key or switch may be triggered by the return to a rearmost position. In the latter two cases, the user is requested to perform a manual priming routine. A manual priming routine will also still be necessary if data pertaining to a setting of the device has been lost due to extraordinary factors affecting the administering device, such as might happen if the dose metering device is turned so rapidly that it can not be detected by a scanning device, or due to mechanical influences.

However, by adopting the method of the present invention, it should be necessary to perform a manual priming routine much less frequently than is the case with the prior art. In addition, the user is prompted to perform one at a time when he is paying more attention to the administering device, such as when changing the product reservoir or after having dropped the device, which means that the user is less likely to miss the prompt to perform the priming routine.

In some embodiments, the absolute nil point represents a starting point from which several doses can then be set. When the first dose is administered, the relative nil point corresponds to the absolute nil point. Whenever a dose is administered, the relative nil point moves farther away from the absolute nil point and does so by the distance travelled by the dose metering device from the reference point for the administering process. The relative nil point and the absolute nil point are preferably stored in a memory in the display unit.

With the aid of the absolute nil point, it is possible to display a total product quantity that has been administered from the reservoir in several consecutive administering processes. To this end, the dose metering distances travelled by the dose metering unit in the direction from the absolute nil point are summed and the sum of the distances travelled by the dose metering device in the metering counter-direction from the absolute nil point is subtracted. The resultant value indicates the effective total quantity of product dispensed since the dose metering device was last set to an absolute nil point. Given that the total volume of product in an ampoule is known, it is also possible to calculate the quantity of product still left in the ampoule on the basis of the total quantity actually dispensed. Accordingly, a message can also be shown on the display, indicating that the contents of the ampoule are running out, for example.

The method proposed by the invention is also used for displays and metering devices with which it is possible to influence the display irrespective of the dose metering device. Electronic displays are advantageously used for this purpose.

The objectives of the present invention are also achieved by a device for administering metered doses of a product. A device of this type has a reservoir for the product, a display unit for displaying a set dosage value and a dose metering device, which dose metering device is moved from an initial position in a metering direction by a dose metering distance in order to set a specific dose of the product. In accordance with embodiments of the invention, the display unit displays the dosage value on the basis of a distance which the dose metering unit has travelled in the metering direction, this distance being defined with effect from a reference point determined by an initial position of the dose metering device for a first dose to be administered or by a position assumed by the dose metering device after administering a dose. The dosage value displayed is therefore the dose corresponding to the distance travelled from the reference point or a relative nil point in the metering direction.

Distances previously travelled in the metering counter-direction are not taken into account in the dose display. The dosage value is therefore obtained by subtracting the distance travelled by the dose metering device in the metering counter-direction, before being moved in the metering direction, from the dose metering distance travelled in the metering direction in order to set the dose.

In such an administering device, the method steps of the method described herein are implemented in accordance with the present invention as a means of displaying and metering doses of the product to be administered in some embodiments, the display is preferably an electronic display and a memory is provided for storing the relative and absolute nil points.

The thinking behind the invention is explained with reference to the example of an injection pen. Naturally, however, any type of administering device may be selected for applying the method, for example infusion devices, inhalers, etc.

### BRIEF DESCRIPTION OF THE DRAWINGS

FIG. 1 is a partial section of one embodiment of a device for administering metered doses of a product in accordance with the present invention, and

FIG. 2 is a diagram illustrating one embodiment of the method in accordance with the present invention.

### DETAILED DESCRIPTION

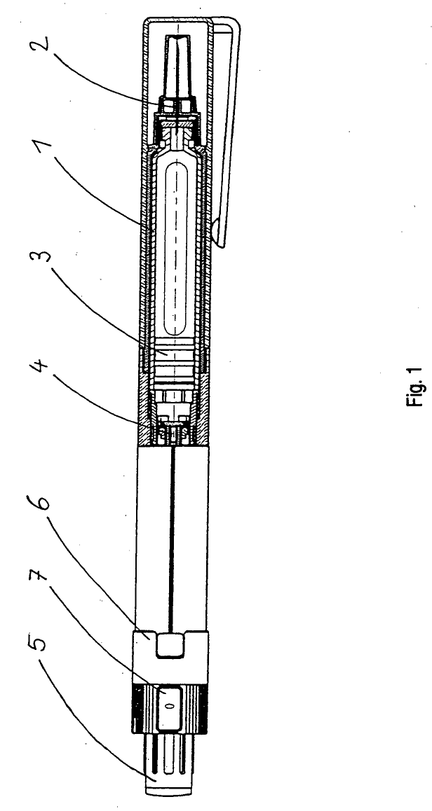

FIG. 1 shows an administering device in the form of an injection pen, which illustrates the principle of a system for administering doses of the type used in the prior art and, in some embodiments, by the invention. In a front region, the injection pen has an ampoule 1 constituting a reservoir for a product or substance to be administered, with an outlet 2. Accommodated in the ampoule is a plunger 3, which can be moved in the direction towards the outlet 2 by means of a drive mechanism. In the example illustrated, the drive mechanism comprises a plunger rod 4 and a drive button 5, by means of which the plunger rod 4 can be moved in the forward direction. Disposed at the rear region of the injection pen is a dose metering device with a rotatable sleeve 6. When the sleeve 6 is turned in the metering direction, a distance can be set which the plunger 4 will travel in the direction towards the outlet 2 when the drive button 5 is operated in order to administer a dose. When the metering sleeve 6 is turned in the metering counter-direction, the plunger rod 4 can be made to cover a nil-dose distance during which the plunger rod 4 does not push the plunger 3 forward when the drive button 5 is operated, which means that no product is forced out of the ampoule 1. The dose metering distance can be set by means of a metering mechanism (not illustrated in FIG. 1), for example, whereby when the metering sleeve 6 is turned, a stop provided for the drive mechanism of the plunger 4 is so disposed that it restricts the drive distance set for the plunger depending on the dose metering distance.

An electronic display unit 7 is provided in the rear region of the injection pen. The display 7 may display the dosage value, for example, due to the fact that contacts disposed on its rear face are operated, for example by means of a camshaft connected to the metering sleeve 6, when the dose is set. The contacts are operated by the camshaft on the basis of the rotation of the metering sleeve and display the dosage setting. The display unit 7 also has a battery, a memory, a computer unit, a display and at least one button to permit manual operation. Other devices and methods of displaying information, e.g., mechanical arrangements or indicia, related to the administration of doses may be used.

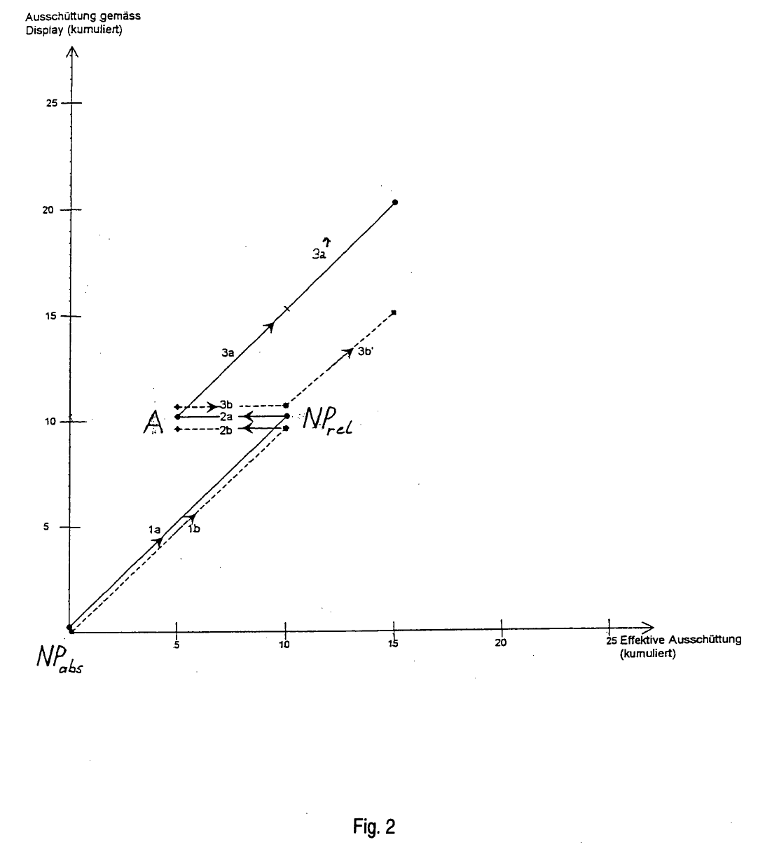

In FIG. 2, the horizontal axis plots the actual dispensing of a product to be administered, i.e., the product quantity actually dispensed, starting from an absolute nil point NP_{abs}. The vertical axis shows dispensing on the basis of a dosage value displayed on the display unit. The units on the axes correspond to the number of discrete dosage units.

The curve plotting metering and a dose display based on the prior art is shown as a continuous line. On the basis of a dose metering distance 1a, a first dose of ten units to be administered is set starting from the absolute nil point NP_{abs} by moving the dose metering device in the metering direction. The value 10 appears on the display unit, as may be seen from the vertical axis. When this set dose is administered, ten product units are forced out of the ampoule. If the injection pen is then incorrectly operated by the user, the dose metering device is then moved in the metering counter-direction by a distance 2a of five dosage units, which means that a dry shot is set, in other words a zero-dosage distance. This displacement in the metering counter-direction triggers a warning on the display, such as a minus sign, for example. Starting from the new initial position A reached due to the displacement of the dose metering device in the metering counter-direction, the user sets a new dose in readiness for administering the product by moving the dose metering device in the metering direction again, along a dose-metering distance 3a. Again, a dose of ten units is set. As soon as the movement in the metering direction starts, the display unit used in the system of the prior art starts to count upwards through these ten units. This means that, starting from the initial position A, a further ten units will be counted. On the vertical axis, therefore, the next ten units will be counted in addition to the first ten units, thereby reaching the value of 20. When the dose-metering distance 3a of 10 units is set, the horizontal axis firstly shows a distance corresponding to the zero-dosage distance 2a of five units which has to be covered before the already administered value of 10 is reached. The next five units are then set along a distance 3a′, equal to the value 15 due to the displacement in the metering direction. Consequently, the dose metering distance 3a is made up of the zero-dosage distance 2a and the distance 3a′. In the case of the prior art, as may be seen from FIG. 2, when the product is administered by the drive mechanism, only five dosage units are dispensed when the second dose is administered because no product is dispensed from the ampoule over the zero-dosage distance 2a. According to the dosage value indicated on the display, however, the second dose corresponds to ten units, which means that a user is unaware of having administered the incorrect dose.

The metering and display system and method in accordance with the present invention are illustrated by a broken line in FIG. 2. Again, a first dose of ten dosage units is set and administered along a dose metering distance 1b starting from the absolute nil point NP_{abs}. The position of the dose metering device after administering this first dose defines a reference point, i.e. a relative nil point NPᵣₑₗ, which is stored in the memory of the display unit. At this stage, it would be possible to perform a manual priming routine, for example if a new ampoule were fitted. In this case, the relative nil point NP_{abs} would correspond to the defined absolute nil point NP_{abs} due to the manual priming.

Once the first dose has been administered and starting from the relative nil point NPᵣₑₗ, the user inadvertently operates the injection pen in the metering counter-direction by five dosage units along the distance 2b, which means that there will be a zero-dosage distance when a dose is administered. In order to set a second dose of ten units, the dose metering device is moved in the metering direction from the resultant initial position A. Accordingly, the zero-dosage distance 3b will initially be covered, corresponding to the distance 2b constituting five units in the metering direction. In accordance with the present invention, during this displacement in the metering direction, the value on the display unit is not counted up by five units to match the displacement. As soon as the dose metering device has reached the position corresponding to the stored relative nil point NPᵣₑₗ, a value “0” is shown on the display unit. Then, as the dose metering device is moved along the distance 3b′ of 5 units, the display counts upwards from the value “0” by 5 units so that it displays the value “5”. As a result of the invention, the display unit therefore shows the dosage value that will actually be administered from this metering position when the drive mechanism is operated. Accordingly, the distance 3b corresponds to an administering distance. The dose metering distance travelled by the dose metering device in the metering direction is made up of the zero-dosage distance 3b and the administering distance 3b′ together. If a user actually wanted to set a dose of ten units, he would have to move the dose metering device by a further five units in the metering direction. He will easily recognise this from the lower value shown on the display unit.

In order to display the total quantity of product administered by the two dosage settings described above, the prior art system sums the two dose-metering distances 1a and 3a with 3a′, which will result in a value of 20 on the display unit and on the vertical axis of FIG. 2, whereas in the case of the present invention, the first dose metering distance 1b covered in the metering direction and the second dose metering distance covered in the metering direction, which is made up of the distances 3b and 3b′, are added and the value of the priming distance 2b or 3b if incorrectly set by the user, is subtracted so that a value of 15 is shown on the display, corresponding to the total quantity of product actually dispensed.

With the aid of the method and the device proposed by the invention, a user is able to control the metering process exactly and ensure that the product is correctly administered. A short dosage can therefore be ruled out.

In the foregoing description, embodiments of the present invention, including preferred embodiments, have been presented for the purpose of illustration and description. They are not intended to be exhaustive or to limit the invention to the precise forms and steps disclosed. Modifications or variations are possible in light of the teachings herein, and all such modifications and variations are within the scope of the invention as determined by the appended claims.

1. A device for administering metered doses of a product, comprising:
a reservoir for the product;
a dose metering device displaceable starting from an initial position in a metering direction along a dose metering distance in order to set a select dose of the product; and
a display unit for displaying a set dosage value, wherein the display unit displays a dosage value on the basis of a distance travelled by the dose metering device in the metering direction, which distance is determined from a reference point constituting a position assumed by the dose metering device after administering a dose or an initial position of the dose metering device in readiness for a first dose to be administered.

2. A method of displaying and metering doses of a product to be administered from a reservoir by means of a dose metering device and a display unit, said method comprising the steps of:
moving the dose metering device starting from an initial position in a metering direction across a dose metering distance to set a selected dose;
defining a reference point by a position of the dose metering device assumed after administering a dose or by an initial position of the dose metering device in readiness for a first dose to be administered; and
displaying a dosage setting as a dosage value on the basis of the distance travelled by the dose metering device from the reference point in the metering direction.

3. A method of displaying and metering doses of a product to be administered from a reservoir by means of a dose metering device and a display unit, said method comprising the steps of:
a) in order to set a specific dose of the product, moving the dose metering device starting from an initial position (NPabs, A) in a metering direction across a dose metering distance (1a; 1b; 3a, 3a′; 3b, 3b′);
b) defining a reference point (NPᵣₑₗ) by a position of the dose metering device assumed after administering a dose or by an initial position (NP_{abs}) of the dose metering device in readiness for a first dose to be administered; and
c) displaying a dosage setting as a dosage value on the basis of the distance (3b′) travelled by the dose metering device from the reference point (NPᵣₑₗ) in the metering direction.

4. The method as claimed in claim 3, wherein the initial position (NP_{abs}) is an absolute nil point and is defined by a position of the dose metering device assumed when:
a reservoir is fitted on the dose metering device,
the display unit has been manually re-set to nil, or
a position of the dose metering device in which it has been moved as far as possible in the metering counter-direction.

5. The method as claimed in claim 3, wherein a total quantity of product administered from the reservoir during administering processes is displayed as the value of the sum of the dose metering distances (1a; 1b; 3a, 3a′; 3b, 3b′;) travelled by the dose metering device from the absolute nil point (NP_{abs}) in the metering direction minus the sum of the distances (2a; 2b) travelled by the dose metering device from the absolute nil point (NP_{abs}) in a metering counter-direction.

6. The method as claimed in claim 3, wherein the reference point (NPᵣₑₗ) corresponds to the absolute nil point (NP_{abs}) before a first dose to be administered is set.

7. The method as claimed in claim 3, wherein, whenever a dose is administered, the reference point (NPᵣₑₗ) for setting a next administering process is at a distance from the absolute nil point (NP_{abs}) in the metering direction constituting the distance (3b′) travelled from the reference point of the administered dosage setting in the metering direction.

8. The method as claimed in claim 3, wherein a warning is displayed by the display unit if the dose metering device is moved by a distance (2a; 2b) in a metering counter-direction.

9. The method as claimed in claim 3, wherein, if the reference point (NPᵣₑₗ) is exceeded as the dose metering device is moved in the metering direction, a code representing the reference point is displayed by the display unit.

10. The method as claimed in claim 3, wherein values to be displayed are displayed by an electronic display unit.

11. The method as claimed in claim 10, wherein at least one of the reference point (NPᵣₑₗ) and the absolute nil point (NP_{abs}) is stored in a memory.

12. The device according to claim 1, wherein a product dose to be administered is displayed and metered using a method comprising the steps of:
moving the dose metering device starting from an initial position in a metering direction across a dose metering distance to set a specific dose of the product;
defining a reference point by a position of the dose metering device assumed after administering a dose or by an initial position of the dose metering device in readiness for a first dose to be administered; and
displaying a dosage setting as a dosage value on the basis of the distance travelled by the dose metering device from the reference point in the metering direction.

13. A device for administering metered doses of a product, comprising:
a) a reservoir for the product;
b) a dose metering device displaceable starting from an initial position (NP_{abs}, A) in a metering direction along a dose metering distance (1a; 1b; 3a, 3a′; 3b, 3b′) in order to set a specific dose of the product; and
c) a display unit for displaying a set dosage value, wherein the display unit displays a dosage value on the basis of a distance (3b′) travelled by the dose metering device in the metering direction, which distance (3b′) is determined from a reference point (NPᵣₑₗ) constituting a position assumed by the dose metering device after administering a dose (NPᵣₑₗ) or an initial position (NP_{abs}) of the dose metering device in readiness for a first dose to be administered.

14. The administering device as claimed in claim 13, wherein a product dose to be administered is displayed and metered using a method comprising the steps of:
moving the dose metering device starting from an initial position (NP_{abs}, A) in a metering direction across a dose metering distance (1a; 1b; 3a, 3a′; 3b, 3b′) to set a specific dose of the product;
defining a reference point (NPᵣₑₗ) by a position of the dose metering device assumed after administering a dose or by an initial position (NP_{abs}) of the dose metering device in readiness for a first dose to be administered; and
displaying a dosage setting as a dosage value on the basis of the distance (3b′) travelled by the dose metering device from the reference point (NPᵣₑₗ) in the metering direction.

15. A method of administering a substance from an administering device comprising a reservoir containing the substance, a dose metering unit and a display unit, the method comprising the steps of:
selecting a dose of the substance to be administered by moving the dose metering unit from an initial position in a metering direction across a dose metering distance;
defining a reference point by an initial position of the dose metering unit in readiness for administering a first dose or a position assumed by the dose metering unit after administering a selected dose; and
displaying the selected dose on a display unit associated with the administering device wherein the displayed dose value is based on the distance travelled by the dose metering unit from the reference point in the metering direction.
